(19) United States
(12) Patent Application Publication
(10) Pub. No.: US 2026/0218304 A1
(43) Pub. Date: Jul. 30, 2026

(54) METHODS AND KITS FOR DIAGNOSING NEURONAL INTRANUCLEAR INCLUSION DISEASE

(71) Applicant: Xiamen Biofast Biotechnology Co., Ltd., Xiamen (CN)

(72) Inventors: Yi-Yi KUO, New Taipei City (TW); Yu-Chiao HSIAO, New Taipei City (TW); Po-Tse LIN, Taipei City (TW); I-Fan CHIU, Hsinchu City (TW)

(21) Appl. No.: 19/457,249

(22) Filed: Jan. 23, 2026

(30) Foreign Application Priority Data
Jan. 24, 2025 (CN) 202510116687.3

(51) Int. Cl. C12Q 1/6883; C12Q 1/6806; C12Q 1/686; G01N 27/447

(52) U.S. Cl. CPC C12Q 1/6883; C12Q 1/6806; C12Q 1/686; G01N 27/44726; C12Q 2600/156

(57) ABSTRACT

Disclosed herein are kits for determining the GGC repeat number of a GGC repeat region in the 5′ untranslated region (5′ UTR) of NOTCH2NLC gene in a nucleic acid sample of a subject. The kit includes first to fourth primers respectively having polynucleotide sequences of SEQ ID NOs: 1 to 4; or first to fourth primers respectively having polynucleotide sequences of SEQ ID NOs: 5, 6, 3 and 4. Also disclosed herein are methods for determining the GGC repeat number of a GGC repeat region in the 5′ UTR of NOTCH2NLC gene in a nucleic acid sample of a subject by use of the present kit, and method for making a diagnosis as to whether the subject has neuronal intranuclear inclusion disease (NIID) based on the determined GGC repeat number of NOTCH2NLC gene.

### REFERENCE TO A SEQUENCE LISTING

The present application is being filed along with a Sequence Listing in electronic format. The Sequence Listing is provided as a file entitled “P4464_SeqList_AF”, created on Jan. 21, 2026, which is 7,177 bytes in size. The information in the electronic format of the Sequence Listing is incorporated herein by reference in its entirety.

### CROSS-REFERENCE TO RELATED APPLICATIONS

This application relates to and claims the benefit of China Application No. 202510116687.3, filed Jan. 24, 2025; the content of the application is incorporated herein by reference in its entirety.

### BACKGROUND OF THE INVENTION

### 1. Field of the Invention

The present disclosure in general relates to the field of disease diagnosis. More particularly, the present disclosure relates to a kit comprising four primers with specific polynucleotide sequences, and uses of the kit in the diagnosis of neuronal intranuclear inclusion disease (NIID).

### 2. Description of Related Art

Neuronal intranuclear inclusion disease (NIID) is a chronic progressive neurodegenerative disease characterized by eosinophilic intranuclear inclusions in the central and peripheral nervous systems as well as in the internal organs. NIID is characterized by a wide range of onset ages and clinical manifestations, such as pyramidal and extrapyramidal symptoms, dementia, convulsions, cerebellar ataxia, peripheral neuropathy, and autonomic dysfunction.

The heterogeneous nature of the disease makes NIID difficult to diagnose. Prior to the identification of genes responsible for NIID, diagnosis is commonly made based on skin biopsies and brain magnetic resonance imaging (MRI), with occasional genetic testing to exclude other genetic disorders e.g., Huntington disease, Alzheimer disease, Multi system atrophy (MSA), spinocerebellar ataxia, and Fragile X syndrome (FXS). Though the first confirmed case of NIID was reported in 1968, yet it was not until the year of 2019 that the GGC repeat expansion in the 5′ untranslated region (5′ UTR) of Notch 2 N-terminal like C (NOTCH2NLC) gene was identified as the genetic cause of NIID. The NOTCH2NLC gene is present at 1q21.1 locus in Human chromosome 1 and encodes a secretory polypeptide, the NOTCH2NLC gene has a structure like that of the N segment of NOTCH2 gene, a result of partially repeating NOTCH2 gene. Examples of homologous genes of NOTCH2NLC gene include NOTCH2NLA, NOTCH2NLB and NOTCH2NLR genes, each has a sequence identity above 99% for a DNA fragment of 100 kb. NOTCH2NLC contains the repeat sequence (GGC)₉, (GGA)₂, or (GGC)₂ in its 5′ UTR, and pathogenic expansion of the GGC repeat in this region has been reported to cause/associate with NIID. The NOTCH2NLC gene of a normal adult typically has a GGC repeat number of less than 40, when the GGC repeat number is more than 60, the NOTCH2NLC gene becomes pathogenic. In general, the NIID subject with clinical manifestation of essential tremor has the GGC repeat number of about 60, the NIID subject with clinical manifestation of Parkinson disease has the GGC repeat number of 80, the NIID subject with clinical manifestation of dementia has the GGC repeat number of 120, while the NIID subject with clinical manifestation of Myasthenia Gravis has the GGC repeat number as high as 200. Further, the presence of GAA or AGC in the GGC repeat region may lower the onset age of NIID, and the presence of GAA or AGC sequence in the GGC repeat region coupling with the GGC repeat number may lead to different clinical manifestations.

Current art uses long-read sequencing (LRS) technique to determine the GGC repeat number in the NOTCH2NLC gene. LRS can directly read long DNA molecules and thereby resolve long repeat expansion regions within genes. Since this approach does not require additional PCR amplification, it is less susceptible to amplification bias associated with GC-rich sequences. However, LRS remains relatively costly. As to repeat-primed PCR (RP-PCR), which is a PCR amplification-based approach for assessing repeat number, the high GC content in the target sequence can make amplification of alleles having a high repeat number difficult.

In view of the foregoing, there exists in the related art a need for an improved method for determining the GGC repeat number in the NOTCH2NLC gene of a subject so as to determine whether such subject has NIID.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a kit for determining the GGC repeat number of a GGC repeat region in the 5′ untranslated region (5′ UTR) of Notch 2 N-terminal like C (NOTCH2NLC) gene. The kit comprises:
a first primer complementary to an upstream sequence of the GGC repeat region in the 5′ UTR of the NOTCH2NLC gene, wherein the first primer comprises a first polynucleotide sequence of SEQ ID NOs: 1 or 5;
a second primer complementary to a downstream sequence of the GGC repeat in the 5′ UTR of the NOTCH2NLC gene, wherein the second primer comprises a second polynucleotide sequence of SEQ ID NOs: 2 or 6;
a third primer complementary to the GGC repeat region in the 5′ UTR of the NOTCH2NLC gene, wherein the third primer comprises a third polynucleotide sequence of SEQ ID NO: 3; and
a fourth primer comprising a fourth polynucleotide sequence of SEQ ID NO: 4;
wherein, the first and fourth primers independently have a fluorophore at its 5′ end.

Examples of fluorophores suitable for use in the present disclosure include, but are not limited to, carboxyfluorescein (FAM), 2′-chloro-7′phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX). According to some embodiments of the present disclosure, the second primer has the fluorophore at its 5′-end. According to other embodiments of the present disclosure, the first and fourth primers independently comprise the fluorophore at its 5′ end.

According to optional embodiments of the present disclosure, the kit further comprises, one or more polymerase chain reaction (PCR) reagents selected from the group consisting of a DNA polymerase, deoxynucleotide triphosphates (dNTPs), betaine, dimethyl sulfoxide (DMSO), glycerol, and ethylene glycol.

According to optional embodiments of the present disclosure, the kit further comprises a plurality of positive controls for constructing a calibration curve for determining the GGC repeat number in the 5′ UTR of the NOTCH2NLC gene, in which each positive control is a vector comprising the NOTCH2NLC gene with 11, 19, 22, 26, 35, 40, 93, or 102 GGC repeats; and a blank control, which is a buffer solution.

The second aspect of the present disclosure pertains to a method for determining the GGC repeat number of a GGC repeat region in the 5′ untranslated region (5′ UTR) of NOTCH2NLC gene in a nucleic acid sample via use of the kit described above. The method comprises:
(a) amplifying the nucleic acid sample with the first, second, third and fourth primers of the present kit in a PCR to produce a plurality of amplicons;
(b) subjecting the plurality of amplicons of step (a) to capillary electrophoresis to separate the plurality of amplicons according to their respective fragment sizes; and
(c) determining the GGC repeat number of each separated amplicon via interpolating its fragment size to a calibration curve established by a plurality of positive controls, in which each positive control is a vector comprising the NOTCH2NLC gene with 11, 19, 22, 26, 35, 40, 93, or 102 GGC repeats;
wherein, in step (a), the concentration of the third primer is at least 500-fold lower than that of the first, second, or fourth primers.

According to some preferred embodiments, in step (a), the first, second, third and fourth primers respectively have the polynucleotide sequences of SEQ ID NOs: 1, 2, 3 and 4.

According to further preferred embodiments, in step (a), the first, second, third and fourth primers respectively have the polynucleotide sequences of SEQ ID NOs: 5, 6, 3 and 4.

A third aspect of the present disclosure pertains to a method of making a diagnosis as to whether a human subject has neuronal intranuclear inclusion disease (NIID) by using the present kit. The method comprises,
(a) obtaining a nucleic acid sample from the human subject;
(b) amplifying the nucleic acid sample with the first, second, third and fourth primers of the present kit in a PCR to produce a plurality of amplicons, in which the concentration of the third primer is at least 500-fold lower than that of the first, second, or fourth primers in the PCR;
(c) subjecting the plurality of amplicons of step (b) to capillary electrophoresis to separate the plurality of amplicons according to their respective fragment sizes;
(d) determining the GGC repeat number of each separated amplicon via interpolating its fragment size to a calibration curve established by a plurality of positive controls, in which each positive control is a vector comprising the NOTCH2NLC gene with 11, 19, 22, 26, 35, 40, 93, or 102 GGC repeats; and
(e) making the diagnosis based on the GGC repeat number determined in step (d), wherein the GGC repeat number of above 60 indicates that the human subject has NIID.

According to certain embodiments of the present disclosure, the concentration of the third primer is 0.5-2.0 nM, and the concentration of the first, second, or fourth primers is 0.5-2.0 μM.

According to embodiments of the present disclosure, the nucleic acid sample is extracted from blood, oral mucosa, body fluids, hair roots, or tissues of the human subject.

According to further embodiments of the present disclosure, the method further comprises administering a symptomatic treatment to the subject to ameliorate or alleviate symptoms of NIID, when the subject has or is the carrier of the NIID.

According to embodiments of the present disclosure, the symptoms of NIID are motor symptoms, seizures, encephalitic/stroke-like episodes, psychiatric symptoms, or ocular symptoms. In some embodiments, levodopa and deep brain stimulation (DBS) are administered to the subject to ameliorate or alleviate motor symptoms. In other embodiments, an anticonvulsant (e.g., carbamazepine) is administered to the subject to ameliorate or alleviate seizures. In further embodiments, a corticosteroid (e.g., glucocorticoid) is administered to the subject to ameliorate or alleviate encephalitic/stroke-like episodes. In still further embodiments, an antipsychotic (e.g., olanzapine) is administered to the subject to ameliorate or alleviate psychiatric symptoms. In other embodiments, an eye drop is administered to the subject to ameliorate or alleviate ocular symptoms.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is an electropherogram depicting the result of an NIID positive sample according to one embodiment of the present disclosure;

FIG. 2 is line graph depicting the calibration curve constructed by NIID positive samples according to one embodiment of the present disclosure;

FIG. 3 is an electropherogram depicting the result of a normal sample #11 isolated from a subject having 15 and 25 GGC repeats in the 5′ UTR of NOTCH2NLC gene determined by PCR solution #1 and primer mixture #1 according to Example 1 of the present disclosure;

FIG. 4 is an electropherogram depicting the result of a normal sample #11 isolated from a subject having 18 and 133 GGC repeats in the 5′ UTR of NOTCH2NLC gene determined by PCR solution #1 and primer mixture #1 according to Example 1 of the present disclosure;

FIG. 5 is an electropherogram depicting the result of a normal sample #94 isolated from a subject having 20 and above 200 GGC repeats in the 5′ UTR of NOTCH2NLC gene determined by PCR solution #1 and primer mixture #1 according to Example 1 of the present disclosure;

FIG. 6 is an electropherogram depicting the result of a normal sample #11 isolated from a subject having 15 and 25 GGC repeats in the 5′ UTR of NOTCH2NLC gene determined by PCR solution #2 and primer mixture #2 according to Example 1 of the present disclosure;

FIG. 7 is an electropherogram depicting the result of a normal sample #11 isolated from a subject having 18 and 133 GGC repeats in the 5′ UTR of NOTCH2NLC gene determined by PCR solution #2 and primer mixture #2 according to Example 1 of the present disclosure; and

FIG. 8 is an electropherogram depicting the result of a normal sample #94 isolated from a subject having 20 and above 200 GGC repeats in the 5′ UTR of NOTCH2NLC gene determined by PCR solution #2 and primer mixture #2 according to Example 1 of the present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “polynucleotide sequence” is understood to mean either a double-stranded DNA or a single-stranded DNA. The polynucleotide sequences of the invention can be isolated, purified (or partially purified), by separation methods including, but not limited to, ion-exchange chromatography, molecular size exclusion chromatography, or by genetic engineering methods such as amplification, subtractive hybridization, cloning, sub-cloning or chemical synthesis, or combinations of these genetic engineering methods.

The term “GGC region” as used herein refers to a region of NOTCH2NLC gene, in which at least two repeats of the GGC sequence are present in the region, in which each GGC sequence and its next GGC sequence may be contiguous (i.e., no intervening nucleotide is present between two GGC sequences) or non-contiguous (i.e., one or more nucleotides are present between two GGC sequences) to each other. According to some embodiments, each GGC sequence and its next GGC sequence form a contiguous sequence without any intervening nucleotide placed therebetween.

As used herein, the term “complementary” refers to the natural binding of polynucleotides under permissive salt and temperature conditions by base-pairing. The binding between polynucleotides is typically formed by hydrogen bonds between nucleotide units in antiparallel polynucleotide, strands or regions. Complementary polynucleotide strands or regions can base pair in the Watson-Crick manner (e.g., A to T, A to U, or C to G), or in any other manner that allows for the formation of stable duplexes. For example, the nucleotide sequence “AGT” binds to the complementary sequence “TCA”. Complementarity between two single-stranded molecules may be “partial”, in which only some of the nucleic acids bind, or it may be complete when total complementarity exists between the single stranded molecules.

The term “diagnosis” as used herein refers to methods by which a skilled artisan can estimate and/or determine the probability (“a likelihood”) of whether or not a subject is suffering from a given disease or condition. In the case of the present invention, “diagnosis” includes using the PCR product amplified by the four primers of the present kit, optionally together with other clinical characteristics, to arrive at a diagnosis (that is, the occurrence or nonoccurrence) of NIID for the subject from which a DNA sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

As used herein, the term “non-human” refers to any animal other than the human species, from whom a sequence (i.e., the non-human sequence of the third primer of the present kit) is obtained (i.e., directly cloning from the non-human animal without any modification) or derived (i.e., cloning from the non-human animal with one or more modifications; such as, replacement or deletion of one or more nucleic acids of the sequence). The non-human animals suitable for obtaining or deriving the present non-human sequence include, but are not limited to, mouse, rat, hamster, guinea pig, rabbit, pig, monkey, sheep, goat, horse, cat, dog, chimpanzee, ape, orangutan, wolf, deer, donkey, zebra, bear, giraffe, lion, and tiger. According to some embodiments of the present disclosure, the non-human sequence of the present kit is derived from bacteriophage without sequence modification.

As used herein, the term “PCR reagents” refers to the chemicals, apart from the specified primers (i.e., the first to the fourth primers of the present kit), needed to perform the PCR process. These chemicals generally comprise four classes of components: (i) an aqueous buffer (also known as PCR buffer), (ii) a water soluble magnesium salt (e.g., MgCl₂), (iii) at least four deoxyribonucleotide triphosphates (dNTPs, including thymidine triphosphate (dTTP), deoxyadenosine triphosphate (dATP), deoxycitidine triphosphate (dCTP) and deoxyguanosine triphosphate (dGTP)), and (iv) a polynucleotide polymerase, preferably a DNA polymerase, more preferably a thermostable DNA polymerase, i.e., a DNA polymerase, which can tolerate temperatures between 90° C. and 100° C. for a total time of at least 10 minutes without losing more than about half its activity. Depending on desired purposes, these chemicals may comprise additional components for improving the efficacy and/or specificity of the PCR process, such as dimethyl sulfoxide (DMSO), betaine, ethylene glycol, and glycerol.

The term “subject” refers to a human species diagnosed by the kit and/or method of the present invention. The term “subject” is intended to refer to both the male and female gender unless one gender is specifically indicated.

### II. Description of the Invention

The present disclosure aims at providing a method of determining the GGC repeat number of a GGC repeat region in the 5′ untranslated region (5′ UTR) of NOTCH2NLC gene in a nucleic acid sample. Accordingly, the present disclosure also encompasses a kit for performing the present method, in which the kit comprises primers for amplifying the GGC repeat region and its upstream and downstream sequences in the 5′ UTR of NOTCH2NLC gene. Based on the GGC repeat number determined by the present methods/kits, whether a subject of the nucleic acid sample has neuronal intranuclear inclusion disease (NIID) is determined. Accordingly, the present disclosure also encompasses a method of treating the subject that has been determined to have NIID by administering to the subject a symptomatic treatment to ameliorate or alleviate symptoms of NIID.

### (i) the Present Kit

As mentioned above, an abnormal expansion of GGC repeats in the 5′ UTR of the NOTCH2NLC gene was identified as the genetic cause of NIID, leading to the recognition of NIID in various neurodegenerative diseases. Thus, the kit of the present disclosure is characterized in having four primers, in which the first and second primers are respectively complementary to upstream and downstream sequences of the GGC repeat region in the 5′ UTR of the NOTCH2NLC gene and independently comprises a non-human sequence at its 5′-end and a mismatch nucleotide at the second or third nucleotides from the last nucleotide at its 3′ end; the third primer is complementary to the sequence in the GGC repeat region and comprises from 5′-end to 3′-end, a non-human sequence and a GGC rich sequence (i.e., a sequence having one or more GGC, CGG, GCG, CCG, or CGC repeats therein); and the fourth primer comprises the non-human sequence of the third primer.

In practice, the first, third, and fourth primers respectively serve as forward primers, which in combination with the second primer that serves as a reverse primer, may amplify various lengths of DNA fragments from a DNA template in a nucleic acid sample. Specifically, the first and second primers are useful in amplifying a first DNA fragment via binding to the upstream and downstream sequences of the GGC repeat region; the polynucleotide sequence of the thus-produced first DNA fragment thus contains, in sequence, the upstream sequence of GGC repeat region, the sequence of GGC repeat region, and the downstream sequence of GGC repeat region. The third and second primers together may amplify a plurality of second DNA fragments via respectively binding to the GGC repeat region and the downstream sequences of the GGC repeat region; the polynucleotide sequences of the thus-produced second DNA fragments thus contain, in sequence, the non-human sequence, the sequence of GGC repeat region, and the downstream sequence of GGC repeat region. The fourth and second primers together may further amplify the second fragments produced by the third and second primers via binding to non-human sequence and the downstream sequence of GGC repeat repeat region of the second DNA fragments. By this way, amplicons respectively having different GGC repeats are produced after amplification. The thus produced amplicons are then subjected to electrophoresis (e.g., a capillary electrophoresis assay) to separate one amplicon from the other via comparing the size of each fragment (e.g., the first DNA fragment) with control fragments (i.e., DNA fragments respectively having known molecular sizes and GGC repeat numbers). According to embodiments of the present disclosure, among the first DNA fragments, every two consecutive DNA fragments or amplicons differ from each other by only 3 base pairs after electrophoresis. As to the second DNA fragments, stutter peaks are found in the electropherogram; accordingly, a skilled artisan may determine the absence or presence of a mutation in the NOTCH2NLC gene (e.g., the abnormal expansion of the GGC repeats, and/or an interruption (e.g., GAA or AGC) in the GGC repeat region) by detecting the stutter peaks, in which the presence of the stutter peaks indicates that the GGC repeat region of the NOTCH2NLC gene comprise a repeat expansion (i.e., an interruption in the GGC repeat).

According to optional embodiments of the present disclosure, the kit further comprises a plurality of positive controls, in which each positive control is a vector comprising the NOTCH2NLC gene with 11, 19, 22, 26, 35, 40, 93, or 102 GGC repeats. After PCR amplification, the positive controls will produce DNA fragments with known GGC repeats, accordingly, a calibration plot may be constructed to reflect the relationship between the sizes of DNA fragments and the GGC repeat numbers. The GGC repeat number of the DNA sample may then be interpolated from the calibration curve. According to one working example of the present disclosure, 4 positive samples are tested, in which the first DNA sample is determined to have a GGC repeat number of 11 and 97; the second DNA sample is determined to have a GGC repeat number of 11, 107, 144 and 151; the third DNA sample has a GGC repeat number of >200, and >200; and the fourth DNA sample has a GGC repeat number of 11, and >200. As would be appreciated, the number of control DNA templates, and the GGC repeating number of each control DNA template should not be limited to the embodiment and example illustrated herein. Any DNA template having a specific number of the GGC repeats may be employed as the DNA template for constructing the calibration curve.

Optionally, the present kit further comprises a negative control, which may be a DNA template that does not have GGC sequence within the polynucleotide sequence thereof. Still optionally, the present kit further comprises a blank control, which does not contain any DNA and preferably is a TRIS buffer solution with a pH value of 8.5.

According to some embodiments of the present disclosure, each of the first, second, third and fourth primers respectively comprise non-human sequences therein and have the polynucleotide sequences of SEQ ID NOs: 1, 2, 3 and 4. In such embodiments, the first and second primers independently comprises, from 5′-end to 3′-end, a non-human sequence of GTTGCTACCCTCGTTCCGA (SEQ ID NO: 7) and a mismatched nucleotide at the second or third nucleotide from the last nucleotide at its 3′-end, thus, the first and second primers respectively have the polynucleotide sequences of SEQ ID NOs: 1 and 2; while third primer comprises, from 5′-end to 3′-end, a non-human sequence of GTAGCCGTTGCTACCCTCGTTCCGA (SEQ ID NO: 4) and a GGC rich sequence (i.e., a sequence having one or more GGC, CGG, GCG, CCG, GCC or CGC), thus the third primer has the polynucleotide sequence of SEQ ID NO: 3; and the fourth primer comprises the non-human sequence of the third primer (i.e., SEQ ID NO:4), so that it may be used to further augment the DNA fragments (e.g., the second DNA fragments as mentioned above) amplified by the third primer. According to other embodiments, neither the first nor the second primers has non-human sequence, accordingly, the first, second, third and fourth primers respectively have the polynucleotide sequences of SEQ ID NOs: 5, 6, 3 and 4. As would be appreciated, the non-human sequence should not be limited to the SEQ ID NOs: 4 or 7 described herein. Instead, any non-human sequence satisfying the functional requirement (i.e., the third and fourth primers comprising the same non-human sequence for amplifying the same DNA fragment in the PCR process) may serve as the non-human sequence for constructing the third and fourth primers of the present kit.

Optionally, for the detection purpose, the primer may have a reporter molecule conjugated at the 5′-end or 3′-end. Non-limiting examples of the reporter molecule suitable to be conjugated with the primer include, but are not limited to, a fluorophore, a luminescent molecule, a phosphorescent molecule, a colorimetric dye, a contrast agent, and an enzyme. According to preferred embodiments of the present disclosure, the first and/or fourth primers independently have a fluorophore conjugated at its 5′-end. Examples of fluorophores suitable for use in the present disclosure include, but are not limited to, carboxyfluorescein (FAM), 2′-chloro-7′phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX). According to some embodiments of the present disclosure, the second primer has a fluorophore at its 5′ end. According to other embodiments of the present disclosure, the first and fourth primers independently comprise a fluorophore at its 5′ end.

According to embodiments of the present disclosure, in addition to the four primers described above, the present kit further comprises PCR reagents for amplifying DNA fragments in PCR assay. In certain examples, the PCR reagents comprise DNA polymerase, PCR buffer (e.g., Tris(hydroxymethyl)aminomethane buffer (Tris buffer), Tris-HCl buffer, or any aqueous buffer known by skilled artisan for conducting the PCR process), MgCl₂, deoxynucleotide triphosphates (dNTPs, including dATP, dTTP, dGTP, and dCTP), dimethyl sulfoxide (DMSO), betaine, ethylene glycol, and glycerol.

### (ii) the Present Methods

Also disclosed herein is a method for determining the GGC repeat number of a GGC repeat region in the 5′ UTR of NOTCH2NLC gene in a nucleic acid sample via use of the kit described above. The method comprises:
(a) amplifying the nucleic acid sample with the first, second, third and fourth primers of the present kit in a PCR to produce a plurality of amplicons;
(b) subjecting the plurality of amplicons of step (a) to capillary electrophoresis to separate the plurality of amplicons based on their respective fragment sizes; and
(c) determining the GGC repeat number of each separated amplicon via interpolating its fragment size to a calibration curve established by a plurality of positive controls, in which each positive control is a vector comprising the NOTCH2NLC gene with 11, 19, 22, 26, 35, 40, 93, or 102 GGC repeats;
wherein, in step (a), the concentration of the third primer is at least 500-fold lower than that of the first, second, or fourth primers in the PCR.

Before commencing the present method, a nucleic acid (i.e., DNA) sample is provided. The nucleic acid sample may be produced by extracting DNA from a cell or tissue of a human subject. The cell or tissue may be any available cell or tissue obtained from the human subject, as long as such cell or tissue contains the DNA of the human subject. For example, the cell may be an epithelial cell, fibroblast, stem cell, blood cell, keratinocyte, or adipocyte. The tissue may be a tissue biopsy, such as a gastric, esophageal, colorectal, brain, hepatic, splenic, or skin biopsy. According to one example of the present disclosure, DNA was extracted from the blood sample of a human subject. The DNA may be extracted from the cell or tissue by use of a commercial kit, or any conventional DNA extraction technique; for example, the phenol/chloroform assay, and detergent (e.g., sodiumdodecyl sulfate, Tween-20, NP-40, and Triton X-100)/acetic acid assay.

Then, the extracted DNA sample is mixed with the first to the fourth primers of the present kit, then amplified by PCR (step (a)). In some preferred embodiments, in step (a), the first, second, third and fourth primers respectively have the polynucleotide sequences of SEQ ID NOs: 1, 2, 3 and 4. In further embodiments, in step (a), the first, second, third and fourth primers respectively have the polynucleotide sequences of SEQ ID NOs: 5, 6, 3 and 4. According to embodiments of the present disclosure, the final concentration of the third primer in the PCR is at least 500-fold lower than the final concentrations of the first, second, and fourth primers. According to certain embodiments of the present disclosure, the final concentration of the third primer is 0.5-2.0 nM, such as 0.5, 0.6, 0.7, 0.8, 0.9, 1.0, 1.1, 1.2, 1.3, 1.4, 1.5, 1.6, 1.7, 1.8, 1.9, and 2.0 nM, and the final concentration of each of the first, second, and fourth primers is 0.5-2.0 μM, such as 0.5, 0.6, 0.7, 0.8, 0.9, 1.0, 1.1, 1.2, 1.3, 1.4, 1.5, 1.6, 1.7, 1.8, 1.9, and 2.0 μM.

The amplicons of step (a) are then subjected to capillary electrophoresis to produce a plurality of separated amplicons based on the fragment size of each amplicon (step (b)). In step (c), the GGC repeat number of each amplicon is determined by interpolating its fragment size into a calibration curve established by a plurality of positive controls. In the present disclosure, the calibration curve is established by a plurality of positive controls, preferably a total of 8 positive controls, in which each positive control is a vector comprising the NOTCH2NLC gene with 11, 19, 22, 26, 35, 40, 93, or 102 GGC repeats. The plurality of positive controls are amplified in the same manner to produce a plurality of amplicons, which are separated by capillary electrophoresis according to their fragment sizes. The calibration curve is constructed by plotting the fragment sizes of the DNA fragments produced from the positive controls against their corresponding GGC repeat numbers. According to one preferred embodiment of the present disclosure, the calibration curve is a liner function of y=2.9343x+167.25, in which y is the fragment size in base pair (bp) of a DNA fragment, and x is the GGC repeat number. Accordingly, once the fragment size of a candidate DNA fragment is known, the GGC repeat number of this candidate DNA fragment may be interpolated from the constructed calibration curve.

Also disclosed herein is a method for making a diagnosis as to whether a human subject has NIID by using the present kit and methods described above. Specifically, the method comprises steps of,
(a) producing a nucleic acid sample from the human subject;
(b) amplifying the nucleic acid sample with the first, second, third and fourth primers of the present kit in a PCR to produce a plurality of amplicons, in which the concentration of the third primer is at least 500-fold lower than that of the first, second, or fourth primers in the PCR;
(c) subjecting the plurality of amplicons of step (b) to capillary electrophoresis to produce a plurality of separated amplicons based on their respective fragment sizes;
(d) determining the GGC repeat number of each separated amplicon via interpolating its fragment size to a calibration curve established by a plurality of positive controls, in which each positive control is a vector comprising the NOTCH2NLC gene with 11, 19, 22, 26, 35, 40, 93, or 102 GGC repeats; and
(e) making the diagnosis based on the GGC repeat number determined in step (d), wherein the GGC repeat number above 60 indicates that the human subject has or is the carrier of the NIID.

In this embodiment, steps (a) to (d) are same as those described above in the method for determining he GGC repeat number of a GGC repeat region in the 5′ UTR of NOTCH2NLC gene in a nucleic acid sample and are not repeated for the sake of brevity.

Once the GGC number of the nucleic acid sample is determined, a clinical practitioner or a skilled artisan may make a diagnosis of NIID based on the determined GGC repeat number. In the case when the GGC repeat number of the nucleic acid sample is determined to be below 40, the human subject is diagnosed as a healthy normal subject. In the case when the GGC repeat number of the nucleic acid sample is determined to be above 60, then the human subject is diagnosed as having NIID.

Accordingly, the method further comprises administering symptomatic treatment to the subject who has or is the carrier of the NIID (i.e., the subject having the GGC repeat number above 60) to ameliorate or alleviate symptoms of NIID.

According to embodiments of the present disclosure, the symptoms of NIID are motor symptoms, seizures, encephalitic/stroke-like episodes, psychiatric symptoms, or ocular symptoms. In some embodiments, levodopa and deep brain stimulation (DBS) are administered to the subject to ameliorate or alleviate motor symptoms. In other embodiments, an anticonvulsant (e.g., carbamazepine) is administered to the subject to ameliorate or alleviate seizures. In further embodiments, a corticosteroid (e.g., glucocorticoid) is administered to the subject to ameliorate or alleviate encephalitic/stroke-like episodes. In still further embodiments, an antipsychotic (e.g., olanzapine) is administered to the subject to ameliorate or alleviate psychiatric symptoms. In other embodiments, an eye drop is administered to the subject to ameliorate or alleviate ocular symptoms

The present kit and method are advantageous in at least the four following aspects:
(1) The first and the second primers are useful in amplifying the 5′ UTR of the NOTCH2NLC gene comprising the GGC region. The second, the third and the fourth primers collectively are useful in amplifying the GGC regions to produce amplicons of various lengths, with every two consecutive amplicons differ from each other by only 3 base pairs. Note that the amplification efficiency of PCR decreases with an increase in the length of the PCR product, thus, when the length of GGC repeat in the GGC repeat region increases, the peak intensity of corresponding amplicon would decrease, thereby producing stutter peaks with two consecutive peaks differing from each other by 3 base pairs. In the case when stutter peaks extending beyond the GGC repeat region of the NOTCH2NLC gene recognized by the first and second primers are found, then larger DNA fragments of the GGC repeat region of the NOTCH2NLC gene are present. Accordingly, stutter peaks may be used to confirm the presence or absence of larger DNA fragments of the GGC repeat region of the NOTCH2NLC gene;
(2) The facts that the final concentration of the third primer is relatively lower than that of the first, second or fourth primers, and the third primer has GC rich sequence and a higher Tₘ ensures that sequences recognized and bound by the third primer would be the first to be amplified and results in the production of amplicons with stutter peaks. Further, as the third primer is present in the lowest concentration, thus it will be the first to be exhausted during PCR and results in the production of amplicons comprising non-human sequence with stutter peaks;
(3) Homologous genes of the NOTCH2NLC gene, such as NOTCH2NLA, NOTCH2NLB and NOTCH2NLR genes, are found in different species and has a sequence identity of >99% in a DNA segment of 100 kb in size, with extremely similar upstream and downstream sequences of the GGC region. Accordingly, the present primers are designed using principles of allele-specific amplification (ASA). In general, a mismatch at or near the 3′ end of a primer can substantially reduce primer extension efficiency by a DNA polymerase, thereby improving amplification specificity. Thus, introducing a mismatched nucleotide at the second or third nucleotide from the 3′ end can increase the specificity for detecting the NOTCH2NLC gene;
(4) In addition to uninterrupted GGC repeats, the GGC repeat region of the NOTCH2NLC gene may contain repeat interruptions or sequence variations, such as GGA, AGG, or other non-GGC motifs. During repeat-primed amplification using the third primer, such interruptions within the GGC repeat region may reduce priming efficiency at the interruption sites and lead to a depressed or discontinuous stutter-peak (ladder) pattern in the electropherogram. Accordingly, in addition to confirming the presence or absence of an expanded GGC repeat region, the stutter-peak pattern may also serve as an indicator of the presence or absence of intervening nucleotides/sequences (i.e., repeat interruptions) within the GGC repeat region, as exemplified by the arrows in FIG. 4;
(5) In the case when the amplicons with higher GGC repeat number (i.e., at least 80% in GC nucleotides) were subjected to capillary electrophoresis, the actual size and the predicted size of the amplicon may differ. If the size is calculated based on the GGC repeat numbers, it would lead to erroneous determination due to the size difference between the actual value and the predicted value. The present kit comprises NIID positive controls independently having 11, 19, 22, 26, 35, 40, 93, 102 GGC repeats, which are used to construct a calibration curve for determining the GGC repeat number in a candidate DNA sample. As the positive controls cover a wide range of GGC repeat numbers, thus could significantly improve the detection accuracy of the present detecting and/or diagnostic methods;
(6) The present kit and methods could quantitatively determine a DNA region rich in GGC sequence, and is the easiest and fast method for accurately determining GGC repeat number for making a diagnosis to NIID in just one PCR reaction, with the result being available in 5 hours. The present method is simple and can be standardized easily.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### PCR Primers

PCR primers for detecting the GGC repeats in the 5′ UTR of Notch 2 N-terminal like C (NOTCH2NLC) gene were synthesized. The polynucleotide sequences of the primers were listed in Table 1.

TABLE 1
The polynucleotide sequences of the present
primers

| | | SEQ |
|---|---|---|
| | | ID |
| Name | Polynucleotide sequence (5′ → 3′) | NO |
| P1 | GTTGCTACCCTCGTTCCGACATTTGCGCCTGTGCTTCGCAC | 1 |
| P2 | GTTGCTACCCTCGTTCCGAGCCCACAGCAGAGCGGCGAAG | 2 |
| P3 | GTAGCCGTTGCTACCCTCGTTCCGAGGCGGCGGCGGCGGC | 3 |
| P4 | GTAGCCGTTGCTACCCTCGTTCCGA | 4 |
| P5 | GGGAGTCGAGGCATTTGCGCCTAT | 5 |
| P6 | CAGCACAGCCAGAGCGCCAGCAGCGCCCACAGCAGAGCTGC | 6 |

Note:
the nucleotide sequence in bold letters is non-human sequence.

### PCR Assay

Two PCR solutions (i.e., PCR solutions #1 and #2) and primer mixtures (i.e., mixtures #1 and #2) respectively comprising the ingredients listed in Tables 2 and 3 were prepared. Then, PCR was conducted by mixing the amplification reagent as listed in Table 4 with a DNA sample (2 μl), a positive control or a blank control (Tris buffer, pH 8.5), then each mixture was amplified under the conditions listed in Table 5. A total of 8 positive controls were used, each positive control was a vector containing NOTCH2NLC gene with 11, 19, 22, 26, 35, 40, 93 or 102 GGC repeats.

TABLE 2
Components of PCR solutions

| | |
|---|---|
| PCR solution #1 | PCR solution #2 |

| | |
|---|---|
| Name | Final concentration |

| | | |
|---|---|---|
| 10X Taq buffer | 1.5 X | 1.5 X |
| dATP | 0.3 mM | 0.3 mM |
| dTTP | 0.3 mM | 0.3 mM |
| dGTP | 0.5 mM | 0.5 mM |
| dCTP | 0.5 mM | 0.5 mM |
| Betaine | 1.5M | 1.5M |
| Ethylene glycol | 1.0M | 1.0M |
| Dimethyl sulfoxide | 3.0% | 3.0% |
| (DMSO) | | |

TABLE 3
Components of primer mixtures

| | |
|---|---|
| Mixture # 1 | Mixture #2 |

| | | | | |
|---|---|---|---|---|
| Primers | P1 | 0.9 μM | P5 | 0.8 μM |
| | P2 | 0.9 μM | P6 | 0.8 μM |
| | P3 | 1.0 nM | P3 | 1.5 nM |
| | P4 | 1.2 μM | P4 | 0.8 μM |

TABLE 4
Components of amplification reagent

| Components | Volume |
|---|---|
| PCR solutions # 1 or #2 | 15.9 μL |
| Primer mixtures #1 or #2 | 2 μL |
| DNA polymerase | 0.1 μL |
| Total volume | 18 μL |

TABLE 5
PCR condition

| | |
|---|---|
| Hold | 95° C./5 minutes |
| 10 cycles | 97° C./30 seconds |
| | 62° C./30 seconds |
| | 68° C./2 minutes |
| 20 cycles | 97° C./30 seconds |
| | 62° C./30 seconds |
| | 68° C./2 minutes and 20 seconds/cycle |
| Hold | 72° C./10 minutes |
| Hold | 4° C./overnight |

After the PCR process, 1.0 μl PCR product was mixed with 0.5 μl LIZ® size standard and 9 μl deionized formamide, the mixture was denatured at 95° C. for 3 min, then immediately placed on ice for 2 min before analyzing by capillary electrophoresis.

### Construction of Calibration Plot

The positive control (i.e., vectors respectively comprising 11, 19, 22, 26, 35, 40, 93 and 102 GGC repeats) was used to construct a calibration plot for the present study. Based on the sizes and the GGC repeat number of the control vectors, a linear regression formula was established: y=2.9343x+167.25, in which x-value represented the GGC repeat number of the DNA molecule, and γ-value represented the size of the DNA fragment (FIG. 2). According to the analytic result, the coefficient of determination (i.e., R²) was 0.9999.

### Stutter Peaks of GGC Repeat Amplicons

In addition to uninterrupted GGC repeats, the GGC repeat region of the NOTCH2NLC gene may contain repeat interruptions or sequence variations, such as GGA, AGG, or other non-GGC motifs. During repeat-primed amplification using the third primer, such interruptions within the GGC repeat region may reduce priming efficiency at the interruption sites and result in a depressed or discontinuous stutter-peak (ladder) pattern in the electropherogram. Accordingly, in addition to confirming the presence or absence of an expanded GGC repeat region, the stutter-peak pattern may also serve as an indicator of the presence or absence of intervening nucleotides/sequences (i.e., repeat interruptions) within the GGC repeat region, as exemplified by the arrows in FIG. 4.

### Example 1: Determination of the GGC Repeat Number of Human Subjects

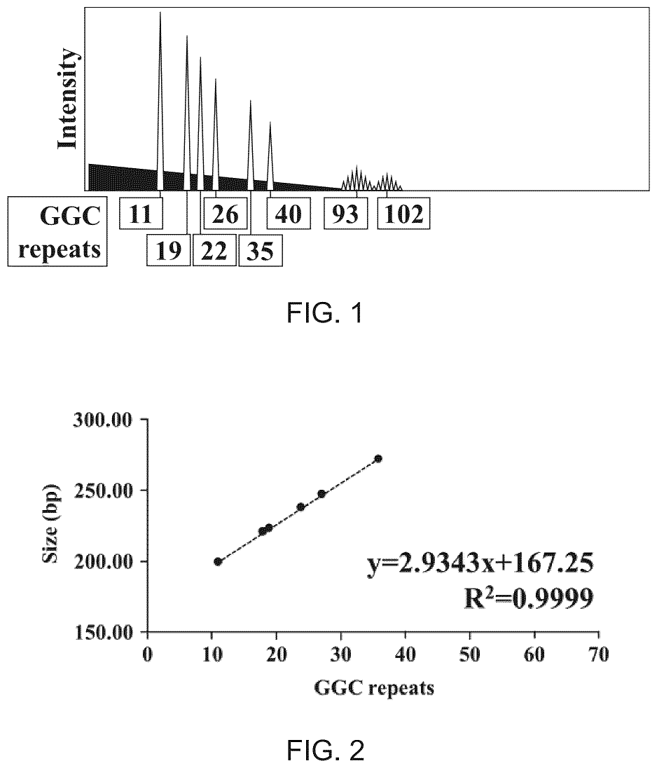

The whole blood samples from 100 subjects suspected of having NIID were obtained with informed written consent. Human genomic DNA was extracted from each whole blood sample by use of a commercial DNA extraction kit. The extracted DNA was diluted to 2.5 to 50 ng/μl and mixed with the present primers and amplified in accordance with the procedure described in “Materials and Methods” section of the present disclosure. Note that the ratio of OD_{260 nm}/OD_{280 nm} was about 1.6-2.0. The identification results were provided in FIGS. 1 and 2 and Table 6.

TABLE 6
Determination of the GGC repeat number of human subjects

| Sample # | Repeat Number | Type |
|---|---|---|
| 1 | 15, 20 | normal |
| 2 | 24 | normal |
| 3 | 22, 24 | normal |
| 4 | 19, 108, 129 | mutation |
| 5 | 15, 20 | normal |
| 6 | 19 | normal |
| 7 | 23, 26 | normal |
| 8 | 21, 26 | normal |
| 9 | 15, 29 | normal |
| 10 | 20, 21 | normal |
| 11 | 15, 25 | normal |
| 12 | 26, 35 | normal |
| 13 | 19, 22 | normal |
| 14 | 22, 31 | normal |
| 15 | 19, 105, 126 | mutation |
| 16 | 15, 92, 168 | mutation |
| 17 | 11, 21 | normal |
| 18 | 16, 25 | normal |
| 19 | 15, 25 | normal |
| 20 | 11, 20 | normal |
| 21 | 19, 141 | mutation |
| 22 | 93, 105 | mutation |
| 23 | 15, 22 | normal |
| 24 | 15, 23 | normal |
| 25 | 16, 22 | normal |
| 26 | 20, 30 | normal |
| 27 | 20 | normal |
| 28 | 13, 27 | normal |
| 29 | 22, 42 | normal |
| 30 | 16, 21 | normal |
| 31 | 15, 26 | normal |
| 32 | 20, 27 | normal |
| 33 | 30, 33 | normal |
| 34 | 16, 124, 160 | mutation |
| 35 | 15, 23 | normal |
| 36 | 22, 34 | normal |
| 37 | 15, 30 | normal |
| 38 | 15, 20 | normal |
| 39 | 15 | normal |
| 40 | 31, 51 | normal |
| 41 | 27, 28 | normal |
| 42 | 17, 21 | normal |
| 43 | 15, 50 | normal |
| 44 | 21 | normal |
| 45 | 23, 24 | normal |
| 46 | 15, 24 | normal |
| 47 | 15, 17 | normal |
| 48 | 41, 116 | Mutation |
| 49 | 40, 128 | Mutation |
| 50 | 25, >200 | mutation |
| 51 | 15, 26 | normal |
| 52 | 15, 30 | normal |
| 53 | 15, 24 | normal |
| 54 | 15, 22 | normal |
| 55 | 16, 21 | normal |
| 56 | 15, 24 | normal |
| 57 | 14, 20 | normal |
| 58 | 15, 17 | normal |
| 59 | 19, 21 | normal |
| 60 | 20, 82, 104 | mutation |
| 61 | 20, 89 | mutation |
| 62 | 15, 93 | mutation |
| 63 | 41, 100, 118 | mutation |
| 64 | 40, 153 | mutation |
| 65 | 15, 30 | normal |
| 66 | 20, >200 | mutation |
| 67 | 16, 22 | normal |
| 68 | 20, 30 | normal |
| 69 | 23, 99 | mutation |
| 70 | 42, 123 | mutation |
| 71 | 20, 22 | normal |
| 72 | 18, 25 | normal |
| 73 | 22, 25 | normal |
| 74 | 15, 122 | mutation |
| 75 | 15, 22 | normal |
| 76 | 14, 20 | normal |
| 77 | 21, 24 | normal |
| 78 | 16, 21 | normal |
| 79 | 15, 26 | normal |
| 80 | 20, 27 | normal |
| 81 | 26, 98, 174 | mutation |
| 82 | 20, 102, 121 | mutation |
| 83 | 13, 25 | normal |
| 84 | 17, 20 | normal |
| 85 | 17, 136 | mutation |
| 86 | 11, 20 | normal |
| 87 | 15, 24 | normal |
| 88 | 15, 23 | normal |
| 89 | 19, 20 | normal |
| 90 | 11, 14 | normal |
| 91 | 25, 123, 139 | mutation |
| 92 | 15, 27 | normal |
| 93 | 21, 92 | mutation |
| 94 | 18 133 | mutation |
| 95 | 15, 24 | normal |
| 96 | 15, 17 | normal |
| 97 | 15, 26 | normal |
| 98 | 15, 24 | normal |
| 99 | 14, 21 | normal |
| 100 | 20, 23 | normal |

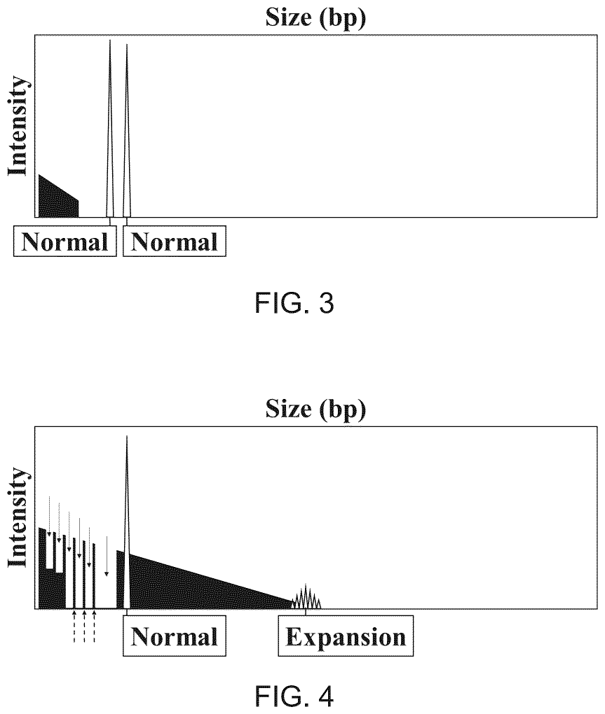

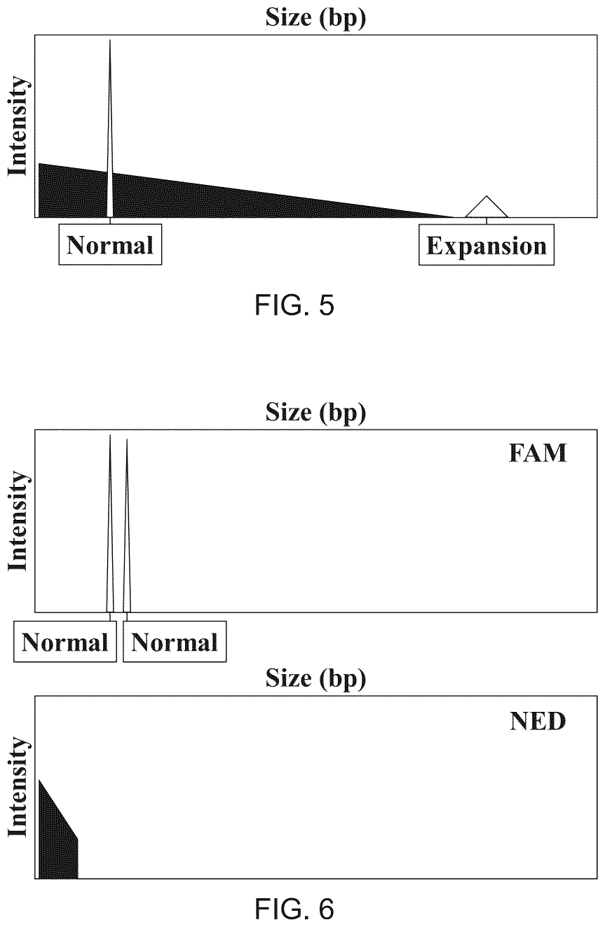

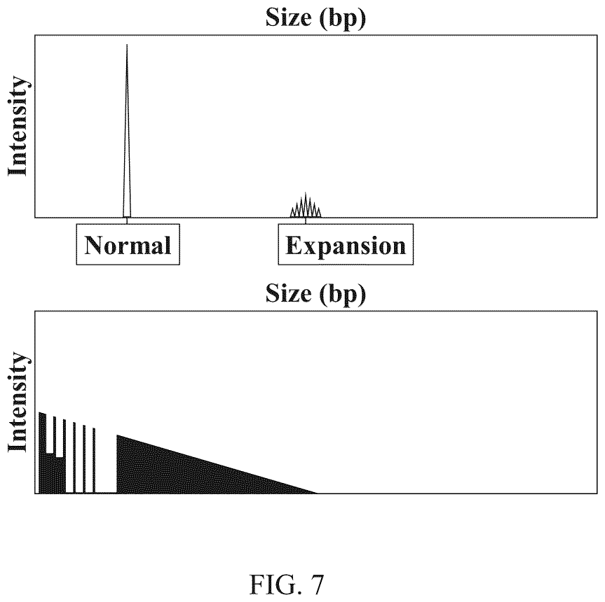

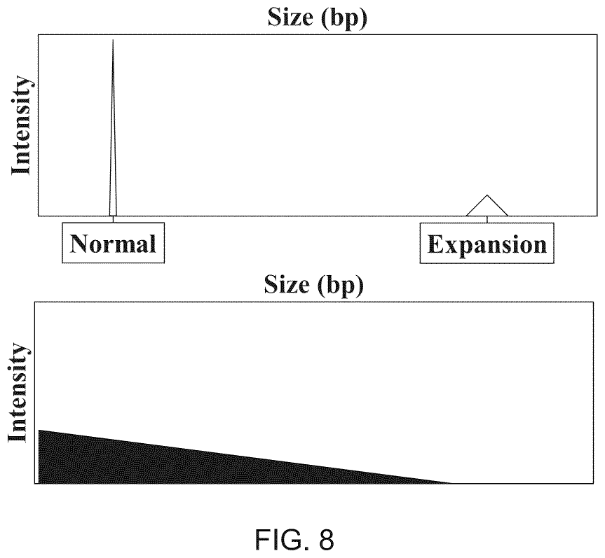

As summarized in Table 6, DNA samples from 23 subjects were determined to comprise abnormal GGC repeat expansions, while the rest 77 DNA samples were determined to be normal. Further, the determination was found to be consistent with that determined by Long-read sequencing (LRS). Also, amplifications performed with the aid of PCR solution #1 and primer mixture #1; or PCR solution #2 and primer mixture #2 all produced same results, in which the results for sample #11 are depicted in FIGS. 3 and 6; the results for sample #66 are depicted in FIGS. 5 and 8; and the results for sample #94 are depicted in FIGS. 4 and 7.

### Example 2: Evaluation of the Diagnostic Accuracy of the Present Kit

### 2.1 Positive Reference Samples

A set of four positive reference samples (PRS #1 to 4) were used to evaluate the accuracy of the present kit and method. Specifically, PRS #1 had GGC repeat numbers of 11 and 97, PRS #3 had GGC repeat numbers of >200 and >200, PRS #4 had GGC repeat numbers of 11 and >200, and PRS #2 had GGC repeat numbers of 11, 107, 144, and 151. The 4 PRSs were respectively diluted to concentrations of 5, 25, and 50 ng/μl, and then mixed with the amplification reagent as described in Materials and Methods of the present disclosure. The GGC repeat number in each PRS was then calculated based on the calibration plot. The data indicated that the accuracy of the present kit/method in determining the GGC repeat number in the PRS samples was 100%.

The detection limit of the present kit/method was evaluated by respectively diluting each positive sample to concentrations of 10, 5, and 2.5 ng/μl, and then mixed with the amplification reagent as described in Materials and Methods of the present disclosure. Tests on each concentration were repeated 20 times. The results confirmed that the detection result for each PRS at each concentration was accurate and consistent.

### 1.2 Negative Reference Samples

In addition to the positive reference samples of Example 1.1, the accuracy of the present kit/method was also evaluated by 6 negative reference samples (NRS #1 to 6). Specifically, NRS #1 to 4 were wild type samples of NOTCH2NLC gene and respectively had GGC repeat numbers of 16 and 16, 17 and 18, 66 and 22, and 11 and 16, while NRS #5 to #6 were non-human genomic DNA samples. Similarly, the 6 NRSs were respectively diluted to concentrations of 5, 25, and 50 ng/μl, and then mixed with the amplification reagent as described in Materials and Methods of the present disclosure. The GGC repeat number in each NRS was then calculated based on the calibration plot. The data indicated that the accuracy of the present kit/method in determining the GGC repeat number in the NRSs was 100%.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A kit for determining the GGC repeat number of a GGC repeat region in the 5′ untranslated region (5′ UTR) of Notch 2 N-terminal like C (NOTCH2NLC) gene comprising:
a first primer complementary to an upstream sequence of the GGC repeat region in the 5′ UTR of the NOTCH2NLC gene, wherein the first primer comprises a first polynucleotide sequence of SEQ ID NOs: 1 or 5;
a second primer complementary to a downstream sequence of the GGC repeat in the 5′ UTR of the NOTCH2NLC gene, wherein the second primer comprises a second polynucleotide sequence of SEQ ID NOs: 2 or 6;
a third primer complementary to the GGC repeat region in the 5′ UTR of the NOTCH2NLC gene, wherein the third primer comprises a third polynucleotide sequence of SEQ ID NO: 3; and
a fourth primer comprising a fourth polynucleotide sequence of SEQ ID NO: 4.

2. The kit of claim 1, wherein the first, second and fourth primers independently comprises a fluorophore at its 5′ end.

3. The kit of claim 2, wherein the fluorophore is selected from the group consisting of carboxyfluorescein (FAM), 2′-chloro-7′phenyl-1,4-dichloro-6-carboxy-fluorescein (VIC), 4,7,2′,4′,5′,7′-hexachloro-6-carboxy-fluorescein (HEX), 6-carboxy-4′-, 5′-dichloro-2′-, 7′-dimethoxy-fluorescein (JOE), 6-carboxytetramethyl-rhodamine (TMR), 2′-chloro-5′-fluoro-7′,8′-benzo-1,4-dichloro-6-carboxyfluorescein (NED), and 5- and 6-carboxy-X-rhodamine (ROX).

4. The kit of claim 3, further comprising:
one or more polymerase chain reaction (PCR) reagents selected from the group consisting of a DNA polymerase, deoxynucleotide triphosphates (dNTPs), betaine, dimethyl sulfoxide (DMSO), glycerol, and ethylene glycol;
a plurality of positive controls; and
a blank control;
wherein,
each positive control is a vector comprising the NOTCH2NLC gene with 11, 19, 22, 26, 35, 40, 93, or 102 GGC repeats; and
the blank control is a buffer solution.

5. A method of determining the GGC repeat number of a GGC repeat region in the 5′ untranslated region (5′ UTR) of NOTCH2NLC gene in a nucleic acid sample, the method comprises:
(a) amplifying the nucleic acid sample with the first, second, third and fourth primers of the kit of claim 1 in a polymerase chain reaction (PCR) to produce a plurality of amplicons;
(b) subjecting the plurality of amplicons of step (a) to capillary electrophoresis to separate the plurality of amplicons based on their respective fragment sizes; and
(c) determining the GGC repeat number of each separated amplicon via interpolating its fragment size to a calibration curve established by a plurality of positive controls, in which each positive control is a vector comprising the NOTCH2NLC gene with 11, 19, 22, 26, 35, 40, 93, or 102 GGC repeats;
wherein,
the concentration of the third primer is at least 500-fold lower than that of the first, second, or fourth primers in step (a).

6. The method of claim 5, wherein the first, second, third and fourth primers respectively have the polynucleotide sequences of SEQ ID NOs: 1, 2, 3 and 4.

7. The method of claim 5, wherein the first, second, third and fourth primers respectively have the polynucleotide sequences of SEQ ID NOs: 5, 6, 3 and 4.

8. The method of claim 5, wherein the kit further comprises,
one or more PCR reagents selected from the group consisting of DNA polymerase, buffer, MgCl₂, deoxynucleotide triphosphates (dNTPs), betaine, ethylene glycol, and glycerol; and
a blank control, which is a buffer solution.

9. The method of claim 5, wherein the nucleic acid sample is extracted from blood, oral mucosa, body fluids, hair roots, or tissues of the human subject.

10. A method of making a diagnosis as to whether a human subject has neuronal intranuclear inclusion disease (NIID) by using the kit of claim 1, comprising,
(a) obtaining a nucleic acid sample from the human subject;
(b) amplifying the nucleic acid sample with the first, second, third and fourth primers of the kit of claim 1 in a polymerase chain reaction (PCR) to produce a plurality of amplicons, in which the concentration of the third primer is at least 500-fold lower than that of the first, second, or fourth primers;
(c) subjecting the plurality of amplicons of step (b) to capillary electrophoresis to separate the plurality of amplicons based on their respective fragment sizes;
(d) determining the GGC repeat number of each separated amplicon via interpolating its fragment size to a calibration curve established by a plurality of positive controls, in which each positive control is a vector comprising the NOTCH2NLC gene with 11, 19, 22, 26, 35, 40, 93, or 102 GGC repeats; and
(e) making the diagnosis based on the GGC repeat number determined in step (d), wherein the GGC repeat number above 60 indicates that the human subject has or is the carrier of the NIID.

11. The method of claim 10, wherein the first, second, third and fourth primers respectively have the polynucleotide sequences of SEQ ID NOs: 1, 2, 3 and 4.

12. The method of claim 10, wherein the first, second, third and fourth primers respectively have the polynucleotide sequences of SEQ ID NOs: 5, 6, 3 and 4.

13. The method of claim 10, wherein the kit further comprises,
one or more PCR reagents selected from the group consisting of DNA polymerase, buffer, MgCl₂, deoxynucleotide triphosphates (dNTPs), betaine, ethylene glycol, and glycerol; and
a blank control, which is a buffer solution.

14. The method of claim 10, wherein the nucleic acid sample is extracted from blood, oral mucosa, body fluids, hair roots, or tissues of the human subject.


SEQUENCE LISTING (7 sequences)

SEQ ID NO: 1 - DNA, 41 bp, synthetic construct
  source 1..41: mol_type = other DNA
gttgctaccctcgttccgacatttgcgcctgtgcttcgcac

SEQ ID NO: 2 - DNA, 40 bp, synthetic construct
  source 1..40: mol_type = other DNA
gttgctaccctcgttccgagcccacagcagagcggcgaag

SEQ ID NO: 3 - DNA, 40 bp, synthetic construct
  source 1..40: mol_type = other DNA
gtagccgttgctaccctcgttccgaggcggcggcggcggc

SEQ ID NO: 4 - DNA, 25 bp, synthetic construct
  source 1..25: mol_type = other DNA
gtagccgttgctaccctcgttccga

SEQ ID NO: 5 - DNA, 24 bp, synthetic construct
  source 1..24: mol_type = other DNA
gggagtcgaggcatttgcgcctat

SEQ ID NO: 6 - DNA, 41 bp, synthetic construct
  source 1..41: mol_type = other DNA
cagcacagccagagcgccagcagcgcccacagcagagctgc

SEQ ID NO: 7 - DNA, 19 bp, synthetic construct
  source 1..19: mol_type = other DNA
gttgctaccctcgttccga